(19) United States
(12) Patent Application Publication
(10) Pub. No.: US 2020/0178879 A1
(43) Pub. Date: Jun. 11, 2020

(54) VAGINAL ELECTRODE

(71) Applicants: Roger Charles Young, Memphis, TN (US); Pretel Inc., Memphis, TN (US)

(72) Inventor: Roger Charles Young, Memphis, TN (US)

(73) Assignee: Pretel Inc., Memphis, TN (US)

(21) Appl. No.: 16/349,570

(22) PCT Filed: Nov. 14, 2017
(86) PCT No.: PCT/US17/61567

§ 371 Date: May 13, 2019

Related U.S. Application Data
(60) Provisional application No. 62/422,184, filed on Nov. 15, 2016.

(51) Int. Cl. A61B 5/00; A61B 5/0448

(52) U.S. Cl. CPC A61B 5/4362; A61B 5/04882; A61B 5/4356; A61B 5/04485

(57) ABSTRACT

The present disclosure discusses a mechanically stabilized sensor. The sensor is configured for intra-vaginal placement and can be used during labor and diagnostic procedures not associated with labor. The sensor can detect bioelectrical signals generated by the uterus and fetal heart. The mechanical stabilization of the sensor to the vaginal mucosa can reduce noise in the bioelectrical signals detected with the sensor. The sensor's stability enables long-term, intra-vaginal recordings to be made.

### CROSS-REFERENCE TO RELATED APPLICATIONS

This application claims priority to U.S. Provisional Patent Application No. 62/422,184 filed on Nov. 15, 2016 and titled “Vaginal Electrode,” which is herein incorporated by reference in its entirety.

### BACKGROUND OF THE DISCLOSURE

Electromyography (EMG) is the measurement of bioelectrical signals of cardiac, skeletal, or smooth muscle. Intra-vaginal recordings can be difficult because movement artifacts can obscure the true EMG signal. It can also be difficult to record long-term recordings.

### SUMMARY OF THE DISCLOSURE

The present disclosure discusses a mechanically stabilized sensor. The sensor is configured for intra-vaginal placement and can be used during labor and diagnostic procedures not associated with labor. The sensor can detect bioelectrical signals generated by the uterus and fetal heart. The mechanical stabilization of the sensor to the vaginal mucosa can reduce noise in the bioelectrical signals detected with the sensor. The sensor's stability enables long-term, intra-vaginal recordings to be made. The sensor can also include safeguards that can enable the sensor to be secured to the target tissue without causing tissue damage. The sensor's compliant materials can reduce tissue damage during ambulation and other movements of the patient.

According to an aspect of the disclosure, a vaginal electrode sensor can include a housing. The housing can include a contact surface that is configured to interface with a vaginal mucosa of a patient. The sensor can include a pedestal that is positioned within an interior volume of the housing. The pedestal can include a first electrode that is substantially flush with the contact surface and configured to contact the vaginal mucosa. The sensor can include a pump. The pump can be configured to generate a vacuum within the interior volume to physically stabilize the first electrode against the vaginal mucosa.

In some implementations, the housing can include a first portion and a second portion. The first portion can include the contact surface. The first portion can include a first elastic modulus. The second portion can be coupled with the first portion. The second portion can include a second elastic modulus that is greater than the first portion.

In some implementations, the pedestal can include a second electrode. The contact surface can include at least one electrode. In some implementations, the contact surface can include a ring electrode. The sensor can include a tether that is coupled with the housing. The tether can enable the placement of the contact surface against the vaginal mucosa. The tether can include a first lumen that can couple the pump with the interior volume of the housing. The tether can include a break-away connector. The break-away connector can disconnect when a predetermined amount of pressure is applied to the tether. The pump can be a syringe.

According to an aspect of the disclosure, a kit can include a vaginal electrode sensor. The vaginal electrode sensor can include a housing. The housing can include a contact surface configured to interface with a vaginal mucosa of a patient. The sensor can include a pedestal positioned within an interior volume of the housing. The pedestal can include a first electrode. The sensor can include a tether coupling the interior volume of the housing with a pump port. The kit can include a pump configured to couple with the pump port.

In some implementations, the pump is a syringe. The pedestal can include a second electrode. The contact surface can include a ring electrode. The kit can include a reference electrode. The housing can include a first portion and a second portion. The first portion can include the contact surface. The first portion can have a first elastic modulus. The second portion can be coupled with the first portion. The second portion can have a second elastic modulus greater than the first portion.

In some implementations, the tether can include a break-away connector that is configured to disconnect when a predetermined amount of pressure is applied to the tether.

According to an aspect of the disclosure, a method to record fetal electrical signals can include positing a vaginal electrode sensor against a patient's vaginal mucosa. The sensor can include a housing. The housing can include a contact surface that is configured to interface with the vaginal mucosa of the patient. The sensor can include a pedestal that is positioned within an interior volume of the housing. The pedestal can include a first electrode. The method can include inducing, with the pump, a vacuum within the interior volume to physically stabilize the first electrode against the vaginal mucosa. The method can include recording, with the first electrode, an electrocardiogram from a fetus. The method can include releasing the vacuum within the interior volume to disconnect the contact surface from the vaginal mucosa.

In some implementations of the method, the housing can include a first portion and a second portion. The first portion can include the contact surface. The first portion can have a first elastic modulus. The second portion can be coupled with the first portion. The second portion can have a second elastic modulus that is greater than the first portion.

In some implementations, the method can include recording a monopolar recording with the first electrode. In some implementations, the pedestal can include a second electrode and the method can include recording a bipolar recording with the first electrode and the second electrode.

### BRIEF DESCRIPTION OF THE DRAWINGS

The figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates a mechanically stabilized sensor.

FIGS. 2A and 2B illustrate different views of the example electrode housing illustrated in FIG. 1.

FIG. 3 illustrates an example method of measuring electrical signals using the sensor illustrated in FIG. 1.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

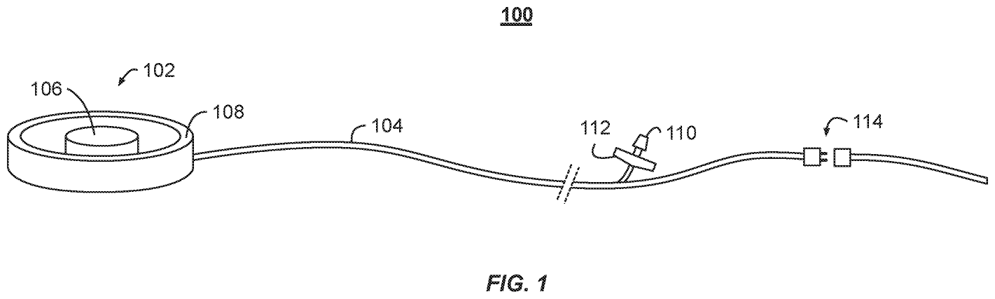

FIG. 1 illustrates an example mechanically stabilized sensor 100. As an overview, the sensor 100 includes an electrode housing 102 and a tether 104 coupled to the electrode housing 102. The electrode housing 102 includes an electrode 106 and a contact surface 108. The tether 104 includes a port 110, a valve 112, and a connector 114.

The electrode housing 102 includes a central electrode 106. The electrode 106 is separated from the wall of the contact surface 108 by a predetermined distance. The face of the electrode 106 can be substantially flush with the contact surface 108. The contact surface 108 can be configured to interface with a patient's vaginal mucosa. For example, the contact surface 108 (or an upper portion of the electrode housing 102) can have an elastic modulus that is similar to that of the vaginal mucosa.

As described below, the volume between the electrode 106 and the wall of the contact surface 108 can be evacuated to generate a vacuum, which, when pressed against target tissue, creates a suction force. The vacuum generated can be any negative pressure differential with respect to the external environment. The vacuum can mechanically stabilize the contact surface 108 and the electrode housing against target tissue, such as the vaginal mucosa. The physical stability provided by the vacuum can reduce or eliminate changes in surface contact, which are known to cause motion artifact in EMG and ECG signal acquisition.

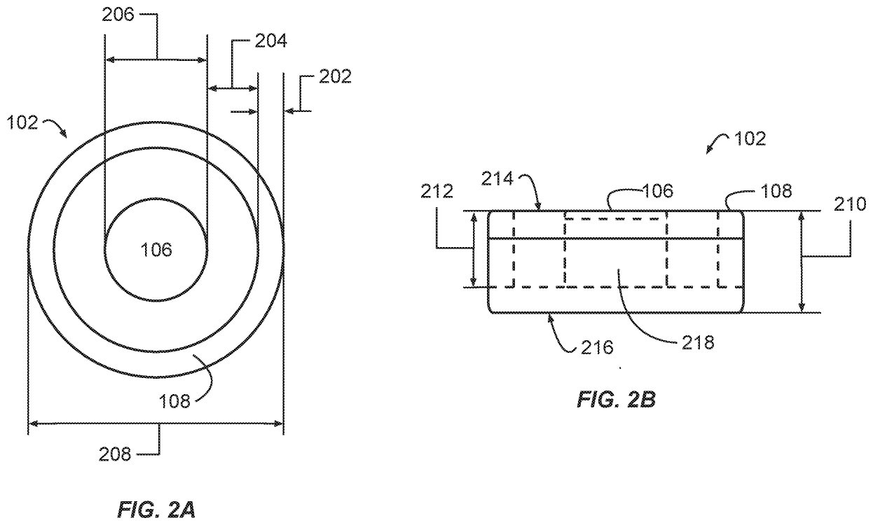

The electrode housing 102 can be constructed from material that has a modulus similar to the modulus of vaginal mucosa. For example, the electrode housing 102 can include liquid silicone rubber (by Proto Labs of Maple Plain, Minn.). The similarity between the modulus of the electrode housing's material and the vaginal mucosa can increase the ability of a seal to be formed between the electrode housing 102 (e.g., the contact surface 108) and the vaginal mucosa surface. A similar modulus can also reduce the risk of damage to the vaginal mucosa. In some implementations, as discussed further in relation to FIG. 2B below, a top portion (also referred to as a first portion) of the electrode housing 102 can be more compliant than a bottom portion (also referred to as a second portion. For example, the first portion can have a modulus similar to the vaginal mucosa. The second portion can be more rigid than the first portion (e.g., have a higher elastic modulus than the first portion) to increase the durability of the electrode housing 102.

As illustrate, the interface surface of the electrode housing 102, electrode 106, and contact surface 108, are each circular in shape. In other implementations, the electrode housing 102, electrode 106, and contact surface 108 can be any combination of oval, square, rectangle, or other shape. The electrode housing 102 can include multiple electrodes 106. For example, the electrode housing 102 can include between 2 and 8 electrodes. In some implementations, the contact surface 108 can include one or more electrodes. The circumference of the contact surface 108 can include a ring electrode. For example, the face of the contact surface 108 can include the electrode material to capture electrical signals from the tissue surrounding the electrode 106. In configurations with multiple electrodes 106, any of the electrodes 106 can be referenced to any of the other electrodes 106 to provide a bipolar (or multipolar) output. The electrodes 106 can also be referenced to a remote ground to provide a monopolar output.

The electrode housing's electrode 106 is an electrode configured to receive bioelectrical signals. The electrode 106 can be disk shaped or can include wound wire. The electrode 106 can include Ag—AgCl, platinum, graphite, other materials suitable for detecting bioelectrical signals, or any combination thereof. The electrode housing 102 can be held in close proximity to a tissue surface (e.g., the vaginal mucosa) by the vacuum generated within the electrode housing 102. For example, vacuum can cause the electrode 106 to be pressed against the vaginal mucosa and substantially remain in contact with the vaginal mucosa during a recording. The generated vacuum can also stabilize the electrode housing 102 against the tissue surface—reducing electrical noise detected by the electrode 106. The electrode 106 senses and transmits the electrical impulses of a patient's body and fetus to an external amplifier, such as that of a patient monitor. The electrical signals are transmitted form the electrode 106 to the amplifier via a wire in the tether 104. In some implementations, the wire is between about 27 G and about 18 G. The wire can be a shielded wire. The wire can be a co-axial wire. The wire can pass from the electrode 106 to the connector 114 through a lumen of the tether 104. In some implementations, the electrode 106 includes a ridged, a honeycombed, or otherwise irregular surface to assist with physical stabilization of the sensor to the vaginal mucosa.

The sensor 100 also includes the tether 104. The tether 104 can include a biocompatible, hollow tube. In some implementations, the wire connecting the electrode 106 to an amplifier runs along the lumen of the hollow tube and in other implementations, the wire runs along an outer surface of the tether 104 or within a wall of the tether 104. The inner diameter of the tether 104 is between about 1 mm and about 2 cm, between about 0.5 cm and about 1.5 cm, or between about 0.5 cm and about 1 cm. The outer diameter of the tether 104 is between about 0.5 cm and about 2 cm, about 1 cm and about 2 cm, or about 1.5 cm and about 2 cm. The tether 104 is configured to have a stiffness high enough to assist with the placement of the electrode housing 102, but flexible enough such that slight movements of the tether 104 do not result in the displacement of the electrode housing 102.

The tether 104 can be between about 20 cm and about 30 cm in length. The tether 104 can include a medical-grade, silicone free, flexible tubing, such as a thermoplastic elastomer. In some implementations, all or a portion of the tether 104 is collapsible. For example, when a negative pressure is applied to the tether 104, the tether 104 can collapse and the volume compliance can provide continuous suction to the cup in the presence of small leaks at the tissue-sensor interface. In some implementations, a portion of the tether 104 near the housing 102 is stiff to enable the tether 104 to be used as a handle for the placement of the housing 102.

The tether 104 also includes a port 110 and valve 112. The port 110 is configured to couple with a syringe or another device capable of drawing a negative pressure of about 150 mmHg with respect to the external environment. In some implementations, the syringe is coupled directly to the port 110 through, for example, a Luer lock type connection. In other implementations, the syringe can be coupled to the port 110 via a length of tubing. The tether 104 can include a lumen that couples the port 110 with the internal volume of the housing (e.g., the space 204). Via the lumen, the pump can generate a vacuum within the space 204. Once a vacuum is generated within the tether 104 and electrode housing 102, the valve 112 enables the retainment of the vacuum. The valve 112 can maintain a negative pressure difference of between about 20 mmHg and about 150 mmHg, between about 50 mmHg and about 100 mmHg, or between about 75 mmHg and about 100 mmHg. In some implementations, the valve 112 maintains the pressure differential below a predetermined level to reduce the likelihood that the vacuum will cause damage to the tissue. The valve 112 can prevent the vacuum from becoming too strong by allowing outside air into the tether 104 if the pressure differential raises above the predetermined threshold.

The tether 104 also includes the connector 114. The connector 114 provides an electrical connection between the sensor 100 and an external device, such as an amplifier that is used to record and monitor the bioelectric signals detected by the electrode 106. The connector 114 can include connection for each electrode 106. The connector 114 can also include one or more connections to ground the wire shielding or other portions of the sensor 100. In some implementations, the sensor's connector 114 is configured to “break-away” from the connector of an amplifier or other device when pulled with a predetermined amount of force. The break-away force of the connector 114 can be less than the force required to remove the electrode housing 102 from the vaginal mucosa once a vacuum is applied to the electrode housing 102. The tether's length between the electrode housing 102 and the connector 114 is such that after a break-away between about 5 cm and about 10 cm protrudes from the vagina. The protruding tether 104 can enable visualization of the sensor should breakaway occur. In some implementations, the break-away can occur when between about 200 g and about 500 g (2 to 5 Newtons) of force is applied to the connector 114. The low break-away force can reduce the chance of injury because the connector 114 will separate from the amplifier before a significant pulling force is applied to the vaginal mucosa. In some implementations, the connector 114 and the port 110 can be combined into a single T-connector.

FIGS. 2A and 2B illustrate different views of the example electrode housing 102 illustrated in FIG. 1. FIG. 2A illustrates a top view of the electrode housing 102 and FIG. 2B illustrates a side view of the electrode housing 102. The width 202 of the contact surface 108 can be between about 1 mm and about 15 mm, between about 5 mm and about 12 mm, or between about 7 mm and about 10 mm. The spacing 204 between the contact surface 108 and the electrode 106 can be between about 3 mm and about 15 mm, between about 5 mm and about 10 mm, or between about 7 mm and about 10 mm. The space 204 can form the internal volume of the housing 102. The pump can remove the gas from the internal volume to form a vacuum that seals the contact surface 108 with the patient's tissue. The diameter 206 (or width) of the electrode 106 can be between about 3 mm and about 25 mm, between about 10 mm and about 20 mm, or between about 15 mm and about 20 mm. The total diameter 208 (or width) of the electrode 106 can be between about 15 mm and about 35 mm, between about 20 mm and about 35 mm, or between about 25 mm and about 30 mm.

FIG. 2B illustrates a side view of the electrode housing 102, with the interior elements illustrated with dotted lines. The height 210 (or thickness) of the electrode housing 102 can be between about 5 mm and about 20 mm, between about 10 mm and about 20 mm, or between about 15 mm and about 20 mm. The depth 212 of the space between the tether 104 and contact surface 108 can be between about 5 mm and about 15 mm or between about 5 mm and about 10 mm.

As discussed above, the electrode housing 102 can include an upper portion and a lower portion. FIG. 2B illustrates the upper portion 214 and the lower portion 216. The upper portion 214 and the lower portion 216 can include different materials, each with a different modulus. In other implementations, the electrode housing 102 is manufactured from a single material and can include a single portion rather than distinct upper and lower portions. As illustrated in FIG. 2B, the electrode 106 is coupled to a pedestal 218. As illustrated, the height of the pedestal 218 is configured such that the top surface of the electrode 106 is substantially flush with the contact surface 108. In other implementations, the height of the pedestal 218 can be configured such that the electrode 106 is raised above the contact surface 108. The material of the pedestal 218 can be compressible such that the pedestal 218 can act like a compression spring to keep the electrode 106 in contact with the tissue surface. In some implementations, the pedestal 218 can include a spring or inflatable balloon that forces the electrode 106 against the tissue surface. The pedestal 218 can include the same material as the upper portion 214. The edges of the electrode housing 102 can be smoothed and rounded to reduce discomfort that may occur with sharp edges. In some implementations, the pedestal 218 can include a plurality of electrode. For example, the pedestal 218 can include a first electrode and a second electrode that can be used for monopolar recordings. In some implementations, when the pedestal 218 only includes a single electrode 106, the recording made with the electrode 106 can be referenced to a second electrode placed distal to the electrode 106. The components of the electrode housing 102 can be manufactured using injection molding, etching, milling, additive manufacturing, or a combination thereof.

In some implementations, the sensor 100 can be a component of a kit. For example, the kit can be a disposable kit that includes each of the components for making an electrical recording from a fetus. The kit can include the sensor 100 described herein. The kit can also include one or more reference electrodes to be used in conjunction with the sensor 100. The kit can also include a syringe or other pump for generating the vacuum that physically stabilizes the electrode 106 to the vaginal mucosa.

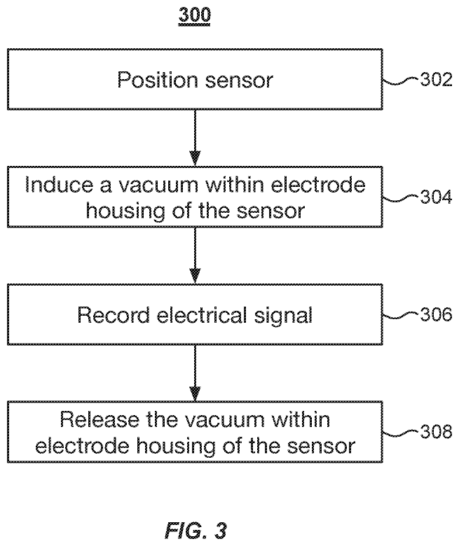

FIG. 3 illustrates an example method 300 of measuring electrical signals. The method includes positioning a sensor (step 302). The method 300 also includes inducing a vacuum within the electrode housing of the sensor (step 304). The method 300 includes recording electrical signals (step 306). The vacuum within the electrode housing of the sensor is then released (step 308) and the sensor can be removed from the patient.

As set forth above, the method 300 includes positing the sensor (step 302). The sensor can be any of the sensors described herein. Manually, a medical professional can place the sensor's electrode intra-vaginally in close approximation to the vaginal or cervical mucosa. In some implementations, the sensor can be placed on the anterior vagina, posterior vagina, or lateral vagina. In some implementations, the sensor can be placed within the rectum. In some implementations, the sensor is positioned prior to child delivery to monitor the bioelectrical signals of the fetus in the uterus and the uterus itself. For example, the sensor can detect the bioelectrical signals generated by the uterus during contractions (and rest) and/or the bioelectrical signals generated by the fetal heart. In some implementations, the method 300 can also include positioning a ground electrode on the patient to be used as a reference for the sensor's electrode. The ground electrode can be positioned on the patient's abdomen, thigh, or side.

The method 300 also includes inducing a vacuum within the sensor's electrode housing (step 304). During the positioning step 302, a syringe (e.g., a syringe between about 10 cc and about 25 cc) can be coupled to the sensor's tether via a check valve. By actuating the syringe, the medical professional can induce a vacuum within the electrode housing. For example, as the medical profession positions the sensor (step 302), the professional can use their other hand to operate the syringe and induce the vacuum. The vacuum generates a suction force that can cause the vaginal mucosa to be drawn into the electrode housing. This can cause the electrode to come into direct contact with the vaginal mucosa. The suction can stabilize the electrode against the vaginal mucosa, which can reduce movement artifact that can be common with EMG and EEG recordings. The tubing of the tether can include a stop cock valve that the profession can rotate once the appropriate level of suction is achieved. The tubing can also be configured to collapse when the appropriate suction is achieved. Once the suction level is fixed, the syringe can be removed from the tether. A portion of the tether that is exterior of the patient can be held against one of the patient's thighs using adjustable straps or tape.

The method 300 also includes recording electrical signals (step 306). Once the sensor is secured to the patient, the sensor can be coupled, via the connector, to an amplifier, signal sender, or other patient monitor. The electrical signals can be bioelectrical signals generated by the patient or a fetus within the patient. In some implementations, the patient monitor can process the received electrical signals and display the signals. The electrical signals can be voltage signals. The patient monitor can also filter the signal—for example, filing out electrical noise or the mother's heartbeat. In some implementations, the electrode detects signals generated by a fetal heart and/or a maternal uterus.

The method 300 also includes releasing the vacuum within the electrode housing (step 308). The vacuum (and suction) can be released by opening the sensor's stop cock or other valve. In other implementations, the positive pressure can be applied to the electrode housing, via the tether, to increase the pressure within the electrode housing and equalize sensor's internal pressure with the environmental pressure.

### CONCLUSION

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A vaginal electrode sensor comprising:
a housing comprising a contact surface configured to interface with a vaginal mucosa of a patient;
a pedestal positioned within an interior volume of the housing, the pedestal comprising a first electrode substantially flush with the contact surface and configured to detect voltage signals from the vaginal mucosa; and
a port to couple with a pump and generate a vacuum within the interior volume to physically stabilize the first electrode against the vaginal mucosa.

2. The sensor of claim 1, the housing further comprising:
a first portion comprising the contact surface, the first portion having a first elastic modulus; and
a second portion coupled with the first portion, the second portion having a second elastic modulus greater than the first portion.

3. The sensor of claim 1, wherein the pedestal comprises a second electrode.

4. The sensor of claim 1, wherein the contact surface comprises at least one electrode.

5. The sensor of claim 1, wherein the contact surface comprises a ring electrode.

6. The sensor of claim 1, further comprising a tether coupled with the housing, the tether configured to enable placement of the contact surface against the vaginal mucosa.

7. The sensor of claim 6, wherein the tether comprises a first lumen coupling the pump with the interior volume of the housing.

8. The sensor of claim 6, wherein the tether further comprises a break-away connector configured to disconnect when a predetermined amount of pressure is applied to the tether.

9. The sensor of claim 1, wherein the pump is a syringe.

10. A kit comprising:
a vaginal electrode sensor comprising:
a housing comprising a contact surface configured to interface with a vaginal mucosa of a patient;
a pedestal positioned within an interior volume of the housing, the pedestal comprising a first electrode; and
a tether coupling the interior volume of the housing with a pump port; and
a pump configured to couple with the pump port.

11. The kit of claim 10, wherein the pump is a syringe.

12. The kit of claim 10, wherein the pedestal comprises a second electrode.

13. The kit of claim 10, wherein the contact surface comprises a ring electrode.

14. The kit of claim 10, further comprising a reference electrode.

15. The kit of claim 10, wherein the housing further comprises:
a first portion comprising the contact surface, the first portion having a first elastic modulus; and
a second portion coupled with the first portion, the second portion having a second elastic modulus greater than the first portion.

16. The kit of claim 10, wherein the tether further comprises a break-away connector configured to disconnect when a predetermined amount of pressure is applied to the tether.

17. A method to record bioelectrical signals comprising:
positing a vaginal electrode sensor against a patient's mucosa, the sensor comprising:
a housing comprising a contact surface configured to interface with the mucosa of the patient; and
a pedestal positioned within an interior volume of the housing, the pedestal comprising a first electrode; and
inducing, with the pump, a vacuum within the interior volume to physically stabilize the first electrode against the mucosa;
recording, with the first electrode, a voltage signal comprising at least an electrocardiogram from a fetus; and
releasing the vacuum within the interior volume to disconnect the contact surface from the vaginal mucosa.

18. The method of claim 17, wherein the housing further comprises:
a first portion comprising the contact surface, the first portion having a first elastic modulus; and
a second portion coupled with the first portion, the second portion having a second elastic modulus greater than the first portion.

19. The method of claim 17, further comprising recording a monopolar recording with the first electrode.

20. The method of claim 17, further comprising referencing the recording of the first electrode with a second electrical recording made by a second electrode positioned on the pedestal.
